(19) United States
(12) Patent Application Publication
(10) Pub. No.: US 2011/0071537 A1
(43) Pub. Date: Mar. 24, 2011

(54) Intramedullary Rod for Assisting Total Knee Joint Replacing Operation and Method for Controle Operation Using the Rod

(76) Inventors: Yoshio Koga, Niigata (JP); Makoto Sakamoto, Niigata (JP); Toshikazu Matsumoto, Niigata (JP); Yuji Tanabe, Niigata (JP)

(21) Appl. No.: 10/594,681

(22) PCT Filed: Mar. 31, 2004
(86) PCT No.: PCT/JP04/04715

§ 371 Date: Sep. 19, 2007

(51) Int. Cl. A61B 17/58; A61B 17/00

(52) U.S. Cl. 606/103; 606/88

(57) ABSTRACT

Disclosed is an intra-medullary rod, including: a cylindrical body made of an X-ray transmitting material; a plurality of wires, which are made of a material that does not transmit X-ray and are deposited at regular intervals along a surface of the cylindrical body in the circumferential direction, being extended in an axial direction in a spiral shape, wherein each of the wires is made in a way that a starting end and a terminating end of the cylindrical body are connected by the shortest distance along the outer surface thereof. In a fluoroscopic image of the cylindrical body, a distance from a reference position to an intersection location of a pair of the wires corresponds to an amount of rotational angle of the intra-medullary rod. By digitizing the intersection location, the rotational angle of the intra-medullary rod is measured.

### TECHNICAL FIELD

The present invention relates to an intra-medullary rod for determining a bone cutting position used in a total knee joint replacement, which is a surgery, and a system and method for assisting the total knee joint replacing operation using the same.

### BACKGROUND ART

The lower limb largely consists of a hip joint, a knee joint, and an ankle joint. Among these, the knee joint that bears much of the load is regarded as the most important joint in human life.

However, the knee joint is easily damaged by injury or a disease since it is subject to constant load yet depending only on ligament. In addition, if the knee is exposed to an overload for a long period of time, the bone may be deformed by its functional adaptation and other hazards might occur. Even though the osteoarthrosis is a typical disease of the knee joint, it is a chronic disease, causing the pain accompanied with the development of a symptom and resulting in gait disturbance.

One of surgical treatment for this disease is a total knee arthroplasty (hereinafter ‘TKA’) in which the entire articular surface is resected and is filled with artificial femoral component, tibial component, and tibia insert. In TKA, a patient's knee area is incised, the tibia is resected, and a total knee joint component (implant) having a synthetic resin member (say, polyethylene polymer) for replacing the joint is mounted on the bone's incisal surface of the tibia. At this time, it is required to perform the tibial incision as much as possible on a surface perpendicular to the anatomical axis of the tibia.

Conventionally, the evaluation of a TKA setting position is generally performed by examining X-ray images of two directions, front and lateral. Since these are 2-dimentional evaluations, if the knee is deformed or there is a bending contracture, it is difficult to regulate the front of the knee for X-ray scanning, consequently affecting the evaluation of the setting position. Because of this, in order to evaluate the setting position accurately, the positional relation among the total joint, the femur, and the tibia should be evaluated three-dimensionally.

Therefore, there have been developed technologies for assisting such TKA, Japanese Patent Laid-Open No. 2003-144454 or 2004-008707 for example, that 3-dimensional simulation related to the setting position is evaluated in a planning stage before intervention.

Moreover, in the planning stage prior to the intervention, a transparent template with calculated magnification was applied to an X-ray image of a patient's knee and the size of a total knee corresponding to the shape of an individual patient and its setting position were planned. During the operation, a rod was inserted into the bone marrow of the patient, and a bone cutting jig prepared in 2-degree unit is connected to the rod in the direction of the setting position. This jig allowed a surgeon to cut the bone according to the plan prior to the intervention.

As to the bone cutting jig, Japanese Patent Laid-Open No. H11-221244 for example disclosed a modular apparatus for total knee joint that is installed at the front end of a femur to be introduced to a bore in the direction of an anatomical axis.

### DISCLOSURE OF THE INVENTION

However, conventional technologies related to TKA posed the following problems:

(1) Estimation of resected bone surface with the intra-medullary rod was possible only in the abduction and adduction directions. Meanwhile, since the estimation was not possible in its rotation and bending directions, a bone cutting device corresponding to each had to be attached and each direction was determined by observation with the naked eye. However, this method made it difficult to determine a precise direction of the resected bone surface, and the surgical instrument has been getting bulky.

(2) The operation assistance using an optical method being currently introduced is mainly focused on the assistance of anterior bone cutting, and the direction of resected bone surface besides the anterior side is not yet known. To find out such resected bone surface, a special machine needs to be provided to an operating room. In addition, to determine the location of an operation field thereof, a control marker needs to be installed in addition to the operation field. Consequently, these act as a hindrance to reducing the operative time or simplification of the surgical instrument.

Therefore, the present invention has been made in view of the above problems, and it is an object of the present invention to provide an intra-medullary rod for assisting total knee joint replacement, which has a marker function and is capable of simply yet precisely recognizing the location or the rotational angle of the intra-medullary rod in a lesion.

It is another object of the present invention to provide an intra-medullary rod incorporated with a bone cutting jig for assisting total knee joint replacement, which has a marker function and is capable of accurately recognizing the information on the intra-medullary rod from a narrow field of vision for fluoroscopy.

It is still another object of the present invention to provide an operational system for assisting bone cutting, which is capable of precisely recognizing the information on an intra-medullary rod from a narrow field of vision for fluoroscopy and has a simplified construction.

It is still another object of the present invention to provide a novel system for assisting bone cutting in connection with TKA, which features an application of 3-dimensional lower limb alignment evaluation system.

It is yet another object of the present invention to provide a system for assisting a total knee joint replacement, which provides a surgeon with accurate information on a resected bone surface of a tibia and is capable of reducing an operative time and simplifying the surgical instrument.

In accordance with an aspect of the present invention, the above and other objects can be accomplished by the provision of an intra-medullary rod, including: a cylindrical body made of an X-ray transmitting material; a plurality of wires, which are made of a material that does not transmit X-ray and are deposited at regular intervals along a surface of the cylindrical body in the circumferential direction, being extended in an axial direction in a spiral shape, wherein each of the wires is made in a way that a starting end and a terminating end of the cylindrical body are connected by the shortest distance along the outer surface thereof.

In accordance with another aspect of the present invention, there is provided an intra-medullary rod comprising a cylindrical body made of a non-metallic material and a plurality of wires in a spiral shape being formed on an outer surface of the cylindrical body at regular intervals, wherein provided that a first circle and a second circle at both ends of the cylindrical body each having a same diameter corresponding to the surface portion are formed, a starting end of each of the wires is located at regular intervals on the first circle, a terminating end of each of the wires is at a position on the second circle rotated in predetermined-degree increments from the starting end, and each of the wires connects the starting end and the terminating end of the cylindrical body by a straight line when the cylindrical body is developed on a plane.

Another aspect of the present invention is that the cylindrical body is made of an acrylic resin and each of the wires is made of stainless steel.

The intra-medullary rod of the present invention is made of stainless steel material of 8 mm in diameter and 150 mm in length for example, in which the rod central portion is made of a metallic core of 3 mm in diameter, the outer side of the core is coated with a cylindrical body made of an acrylic material, thereby forming a cylindrical body being 8 mm in external diameter. In addition, four stainless steel wires each being 1 mm in diameter are inclinedly buried and fixed at regular intervals of 90 degrees in the surface of the cylindrical body made of this acrylic resin.

Another aspect of the present invention provides a bone cutting positioning jig for indicating a bone cutting direction, which comprises: a bone cutting direction indicator having a base, a universal joint being movably and rotatably supported around three axes to the base of the bone cutting direction indicator through a ball joint and having a direction indicating jig, and an intra-medullary rod fixed to one end of the universal joint, wherein the intra-medullary rod comprises a cylindrical body made of a non-metallic material and a plurality of wires in a spiral shape being formed on an outer surface of the cylindrical body at regular intervals, in which, provided that a first circle and a second circle at both ends of the cylindrical body each having a same diameter corresponding to the surface portion are formed, a starting end of each of the wires is located at regular intervals on the first circle, a terminating end of each of the wires is at a position on the second circle rotated in predetermined-degree increments from the starting end, each of the wires connects the starting end and the terminating end of the cylindrical body by a straight line when the cylindrical body is developed on a plane, and an intersection of each of the wires has a marker indicator function providing rotational position information; wherein the bone cutting direction indicator comprises a guide having a guide groove on an upper surface and being attached to the base through a shaft for determining an inversion and evagination angle, and a guide having a guide groove on an upper surface and being attached to the base through a shaft for determining a bending and stretching angle; wherein a front end of a direction indicating jig of the universal joint is inserted into an intersection of the groove of the guide for determining an inversion and evagination angle and the groove of the guide for determining a bending and stretching angle; and wherein the bone cutting direction is determined by moving the inversion and evagination angle-determining guide of the direction indicating jig to indicate the inversion and evagination angles and by moving the bending and stretching angle-determining guide to indicate the bending and stretching angles.

Still another aspect of the present invention provides a total knee joint replacement assisting terminal, which uses a computer, has a planning assistance function before an intervention and an intraoperative assistance function, and supports a total knee joint replacement performed with aid of a bone cutting positioning jig indicating a bone cutting direction by the intraoperative assistance function, wherein the bone cutting positioning jig comprises a bone cutting direction indicator having a base, a universal joint being supported to be movable and rotatable around three axes to the base of the bone cutting direction indicator through a ball joint and having a direction indicating jig, and an intra-medullary rod fixed to one end of the universal joint; wherein the intra-medullary rod comprises a plurality of wires, which are made of a material that does not transmit X-ray and are deposited at regular intervals along a surface of the cylindrical body in the circumferential direction, being extended in an axial direction in a spiral shape; wherein each of the wires is made in a way that a starting end and a terminating end of the cylindrical body are connected by the shortest distance along the outer surface thereof; wherein the intraoperative assistance function includes: a function of acquiring, by a C-arm fluoroscopic apparatus, a radiographic image data of the intra-medullary rod inserted into a tibia; a function of acquiring, on the fluoroscopic image obtained by the fluoroscopic apparatus, rotational position information of the intra-medullary rod in a medullary cavity out of the position of an intersection of a pair of wires of the intra-medullary rod; and a function of determining a bone resection margin using the intra-medullary rod as a reference anatomical axis; and wherein an anterior articular surface of the femur is determined perpendicularly to a load shaft from an angle with the femoral load shaft determined using the planning before the intervention, and a resected bone surface is determined.

According to the method for controling an operation of the present invention, in the planning before the intervention, the bone shape of each patient is made to a 3-dimensional model in the alignment evaluation system, an anatomical coordinate system is established, and a total joint positioning plan according to the location of a total joint shaped model can be set. Next, the intra-medullary rod is inserted into a lesion of a patient during the operation, and two-directional X-ray fluoroscopic images are taken using the C-arm fluoroscopic apparatus, thereby 3-dimensionalizing the bone shape. The position alignment is performed based on the plan before the intervention, and a resected bone surface about the direction of the intra-medullary rod is calculated based on the total joint setting position. Meanwhile, a bone cutting jig is connected to the intra-medullary rod via the universal joint. Using the bone cutting direction indicator, the direction of the bone cutting jig is determined to coincide with the resected bone surface obtained from the calculation.

According to the present invention, it becomes possible to accurately recognize the direction of a rod from a narrow field of vision for fluoroscopy, by using the special bone cutting positioning jig employing the intra-medullary rod. The coordinate system (exclusive of rotation) of the intra-medullary rod is determined by digitizing, with help of the intra-medullary rod of the present invention, both ends of the intra-medullary rod with the C-arm fluoroscopic image during the operation. Next, by digitizing the intersection of steel wires buried in the cylindrical body made of a X-ray transmitting material of the intra-medullary rod, a distance from the reference position in the axial direction is obtained, and a rotational angle of the intra-medullary rod corresponding to this distance can be measured.

Moreover, it is now possible to calculate the direction and amount of the back and forth, lateral and far-to-near movement of the bone cutting jig about the resected bone surface in the planning before the intervention. Only medical instruments available in normal medical facilities are used, leading to reduction in operative time, and simplification of surgical instruments to be cleaned.

### BRIEF DESCRIPTION OF THE DRAWINGS

The above and other objects, features and other advantages of the present invention will be more clearly understood from the following detailed description taken in conjunction with the accompanying drawings, in which:

FIG. 1 are schematic views of a system for assisting a total knee joint replacement according to one embodiment of the present invention, in which FIG. 1A shows a construction of the system and FIG. 1B is an explanatory view of functions;

FIG. 2 are schematic views of an intra-medullary rod according to one embodiment of the present invention, in which FIG. 2A is a front view of the intra-medullary rod; FIG. 2B is a left lateral view of the intra-medullary rod; FIG. 2C is a cross-sectional view taken along line C-C in FIG. 2A; FIG. 2D is an enlarged view of a cylindrical body of the intra-medullary rod; FIG. 2E is a plan development figure of an outer surface of the cylindrical body; and FIG. 2F is a diagram illustrating a relation between a rotational angle of the intra-medullary rod and a distance from a pair of continuous lines to an intersection;

FIG. 3 is a schematic view of a universal joint indicating the direction of bone cutting, in which FIG. 3A is a front view of the universal joint; FIG. 3B is a side view of the universal joint; FIG. 3C is a top view of the universal joint; FIG. 3D is a bottom view of the universal joint; and FIG. 3E is a perspective view of the universal joint;

FIG. 4 are schematic views of a bone cutting direction indicator, in which FIG. 4A is a perspective view of the bone cutting direction indicator; FIG. 4B is a plan view of the bone cutting direction indicator; FIG. 4C is a front view; FIG. 4D is a right lateral view; FIG. 4E is a cross-sectional view taken along line A; and FIG. 4F is a plan view of an indicator probe;

FIG. 5 is a diagram explaining an overall construction and operation of a bone cutting positioning jig;

FIG. 6 are explanatory views of a knee joint, in which FIG. 6A is a diagram showing a relation between the shape of a knee joint and the movement; and FIG. 6B is a diagram showing a femoral tibia angle or functional axis;

FIG. 7 is a flow chart describing a planning assistance process before an intervention;

FIG. 8 are photographs for explaining a planning before the intervention using a 3-dimensional limb alignment evaluation system, in which FIG. 8A shows a bone deformation treatment process; and FIG. 8B shows a limb alignment calculation process;

FIG. 9 is a flow chart describing an intraoperative assistance process;

FIG. 10 is a photo showing an example of a C-arm fluoroscopic image an intra-medullary rod;

FIG. 11 is a diagram for explaining a mounting state of a total joint component (implant); and

FIG. 12 are photographs for explaining an effect of the present invention, in which FIG. 12A is a photograph showing an example of a C-arm fluoroscopic image in case that an intra-medullary rod with a marker function of the present invention is used; and FIG. 12B is a photograph showing an example of a C-arm fluoroscopic image in case that an attachment type intra-medullary rod besides the marker of a comparative example.

### BEST MODE FOR CARRYING OUT THE INVENTION

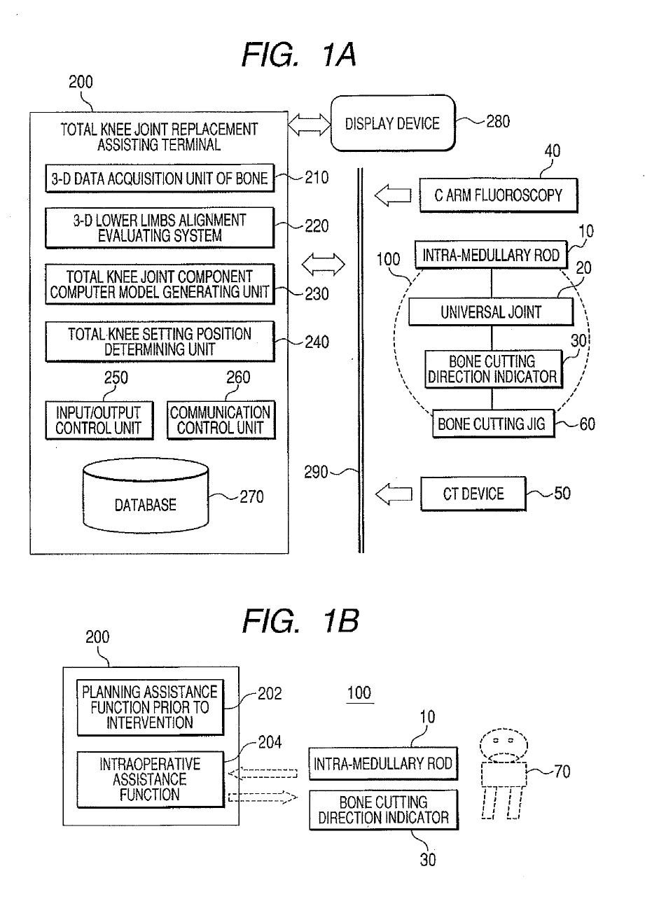

Referring to FIG. 1, the following will now explain a bone cutting positioning jig and a system for assisting a total knee joint replacement using the same, in accordance with one embodiment of the present invention. As shown in a configuration of the system in FIG. 1A, the system for assisting a total knee joint replacement of the present invention includes a bone cutting positioning jig 100 and a total knee joint replacement assisting terminal 200.

First, the bone cutting positioning jig 100 includes an intra-medullary rod 10, and a bone cutting direction indicator 30 connected to the intra-medullary rod via a universal joint 20. A bone cutting jig 60 is mounted on the bone cutting positioning jig 100. The intra-medullary rod 10 is inserted into a medullary cavity in the knee joint of a patient during a total knee joint replacement, and is used to determine a bone resection margin with respect to the axis as a referential anatomical axis.

Meanwhile, the total knee joint replacement assisting terminal 200 constituted by a computer has functions of the CPU, memory, memory device, input/output control units and communication control, and processes various information necessary to assist a planning before the intervention and to provide an intraoperative assistance by loading a program stored in a memory device on a memory and executing the program.

The total knee joint replacement assisting terminal 200 includes a bio 3-dimensional bone data acquisition unit 210, a 3-dimensional limb alignment evaluation system 220, a total knee joint component computer model generation unit 230, and a total knee joint mount positioning processor 240. It further includes a communication control unit 260, a database 270, and a display device 280. An operation panel of the display device is a touch panel and has a graphical user interface (GUI) function. As an operator commands and operates a dot or icon on the operation panel with a pointing device, such as, a mouse or a pen, an input process can be performed to the total knee joint replacement assisting terminal 200.

The total knee joint replacement assisting terminal 200 is connected to a C-arm fluoroscopic apparatus 40 or a CT scanner 50 through a communication network 290. A radiographic image data of a patient's tibia taken by the C-arm fluoroscopic apparatus 40 before the intervention and during the operation is inputted to the total knee joint replacement assisting terminal 200. Moreover, the total knee joint replacement assisting terminal 200 is capable of communicating with other medical information systems, e.g., an electronic Karte system, through the network 290.

As depicted in the view of functional explanation in FIG. 1B, the total knee joint replacement assisting terminal 200 has a planning assistance function 202 prior to an intervention and an intraoperative assistance function 204 which are realized by properly using each constituent shown in FIG. 1A.

In the total knee joint replacement, the planning assistance function 202 prior to an intervention means assisting a planning process for mounting a total knee joint (implant) on the resected bone surface of a tibia. To begin with, an image data of a tibia which is the bone cutting target of a patient 70, obtained by the C-arm fluoroscopic apparatus 40 is acquired. For the image data, a CT image or MRI image data can be used.

From the 3-dimensional radiographic image data thus acquired, a load shaft of the patient's foot is determined. In addition, based on the respective 3-dimensional data of the acquired radiographic image data and the data of the implant's shape to be mounted, a 3-dimensional simulation for mounting the implant is performed. Each of the 3-dimensional data and the data on the total knee joint mount position obtained from the 3-dimensional simulation are recorded and kept in the database 270.

The intraoperative assistance function 204 of the total knee joint replacement assisting terminal 200 involves determining a bone resection margin using the intra-medullary rod 10 inserted into a medullary cavity of a lesion of the patient 70 as a referential anatomical axis. That is to say, the intra-medullary rod 10 is inserted into the patient's femur, and a radiographic image data thereof is obtained by the C-arm fluoroscopic apparatus 40. As the intra-medullary rod 10 has a marker indicator function and thus, is capable of obtaining the rotational position of the intra-medullary rod 10 in the medullary cavity. By digitizing both ends of the intra-medullary rod 10 on the fluoroscopic image taken by the C-arm fluoroscopic apparatus 40, the coordinate system of the intra-medullary, except for the rotation, are determined. Later, rotation is determined with the rotational position information by the marker indicator function of the intra-medullary rod 10. Then, an anterior articular surface of the femur is determined perpendicularly to the load shaft from an angle with the femoral load shaft determined in the planning before the intervention, and a resected bone surface is determined. To be in correspondence to this resected bone surface, an angle of the bone cutting jig 60 is set by the bone cutting direction indicator 30, and resection is done with a bone saw. Further details on this will be provided later.

The following will now explain in detail about the bone cutting positioning jig 100 which is one of the features of the present invention.

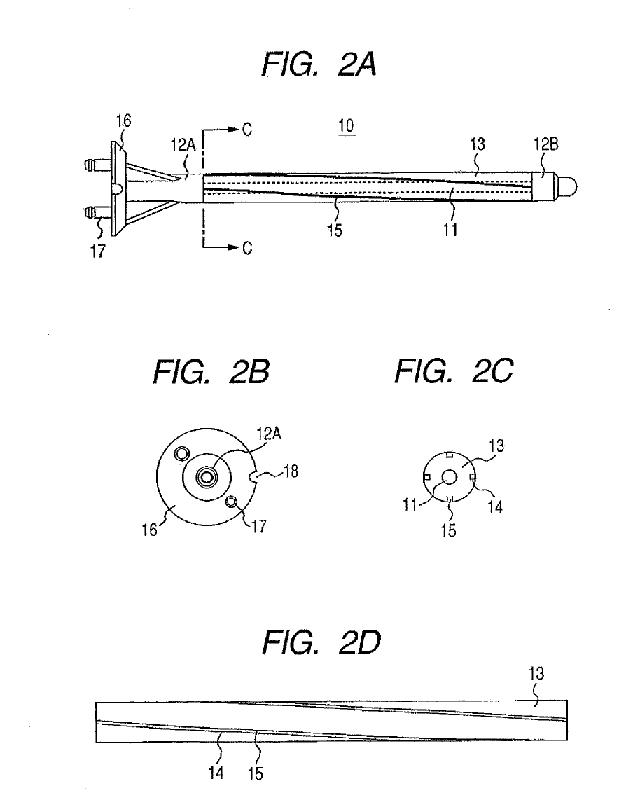

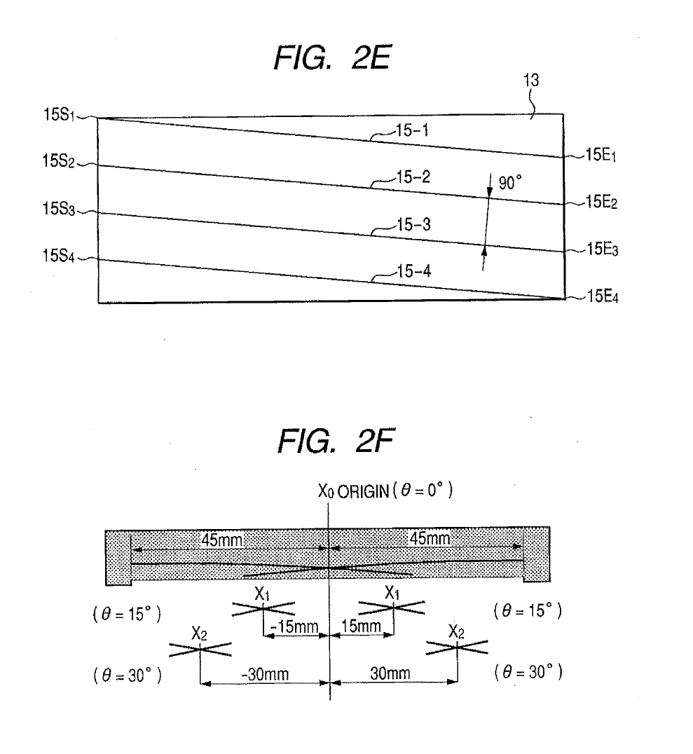

First of all, the intra-medullary rod 10 is discussed referring to FIG. 2. FIG. 2A is a front view of the intra-medullary rod, FIG. 2B is a left lateral view of the intra medullar rod, and FIG. 2C is a cross-sectional view taken long line C-C in FIG. 2A.

The intra-medullary rod 10 is constituted by a metallic core 11, a pair of both ends 12A and 12B connected with the metallic core as one body, a cylindrical hollow body 13 fixed at the central portion between the both ends and around the circumference of the metallic core and extended in an axial direction, and four spiral wires 15 buried in a groove 14 on the outer surface portion of the cylindrical body. The cylindrical body 13 is made of an X-ray transmitting material, while the wires 15 are made of a material that does not transmit X-ray. One side end 12A of the intra-medullary rod has a metallic flange 16, and the flange 16 has two pins 17 to be connected to the universal joint 20. Moreover, reference numeral 18 denotes a notch showing the location of abase line of the rotation direction of the intra-medullary rod. The front end 12B of the intra-medullary rod is also made of a metallic material, and has a small diameter to be easily inserted.

A material of the intra-medullary rod 10 needs to have a predetermined mechanical strength and, at the same time, needs to be biochemically stable in vivo so that it does not adversely affect a human body when inserted into a bone marrow. In addition, a material of the cylindrical hollow body 13 should be able to transmit X-rays. Examples of the material satisfying such condition include an acrylic resin and other x-ray transmitting type resins.

The spiral wires 15 are made of stainless steel for example and a plurality of the wires are buried in the outer surface of the cylindrical body at regular intervals. In addition, the flange 16 is made of stainless steel. The metallic core 11, the flange 16, and the front end 12B of the intra-medullary rod may be formed as one body.

FIG. 2D is an enlarged view of the cylindrical body 13 of the intra-medullary rod, and FIG. 2E is a plan development figure of an outer surface of the cylindrical body. Suppose that there are two circles (circle 1 and circle 2) having the same diameter from both ends of the cylindrical body 13 to the outer surface. Each starting end 15S (15S1, 15S2, 15S3, and 15S3) of the wires 15 (15-1, 15-2, 15-3, and 15-4) are set up on the circle 1 at intervals of 90 degrees. Meanwhile, each terminating end 15E (15E1, 15E2, 15E3, and 15E4) of the wires 15 are set up on the circle 2 at a 90 degree rotated position from the starting end. Each wire 15 is formed in a manner that the starting end and the terminating end of the cylindrical body are connected by the shortest distance along the outer surface of the cylindrical body. In other words, when the outer surface of the cylindrical body 13 of the intra-medullary rod is spread out, a groove is formed along the straight line connecting the starting end to the terminating end located at a 90 degree position from the starting end, and the lines 15 are placed in this groove.

An embodiment of a more detailed construction of the intra-medullary rod 10 is now illustrated. The intra-medullary rod 10 is substantially cylindrical in shape, approximately 8 mm in diameter and 150 mm in length for example. The central portion of the rod is made of a stainless steel material core having a diameter of 3 mm, and both ends of the rod are also made of a stainless steel material. The outer side of the core is covered with a cylindrical shape cover made of an acrylic material, thereby forming a cylindrical body being 8 mm in external diameter having 90 mm of length in an axial direction. In addition, four stainless steel wires each being 1 mm in diameter are buried and fixed at regular intervals of 90 degrees in the surface of the cylindrical body made of this acrylic resin in a spiral shape. Moreover, each wire is connected to the ends 12A and 12B in advance and it can be molded with an acrylic resin. Besides the stainless steel, other metallic materials such as a titan alloy and the like can be used as well.

The intra-medullary rod 10 of the present invention has a marker function. The following will now explain this marker function. When the intra-medullary rod 10 is photographed by the C-arm fluoroscopic apparatus 40, an X-ray transmits the cylindrical body 13 made of an acrylic material and thus, only the steel wires 15 buried in its circumference are photographed. This fluoroscopic image shows an intersection of a pair of steel wires, i.e., the steel wire in the front of the cylindrical body and the steel wire in the rear of the cylindrical body. When the intra-medullary image in a horizontal status is photographed, the location of the intersection horizontally moves on the base line in the axial direction accompanying the rotation of the intra-medullary rod. In addition, in case that there are four steel wires, although the intersection on the fluoroscopic image is shown in two groups, it is sufficient to focus on one of them. Therefore, for the fluoroscopic image, by digitizing the intersection of a pair of steel wires on the base line, the reference position of the cylindrical body 13, for example, a distance from the center of the cylindrical body 13 in the axial direction is obtained, and a rotational angle of the intra-medullary rod can be measured out of this distance. For instance, a fluoroscopic image is placed on the panel of a display device 280 and the like, and the operator commands/operates with a mouse or a pen the intersection of the fluoroscopic image, so that the data of the rotational angle of the intra-medullary rod about the total knee joint replacement assisting terminal 200 can be inputted. Also, the rotational angle data of the intra-medullary rod may be obtained by image processing the fluoroscopic image in the total knee joint replacement assisting terminal 200 and then by obtaining the intersection on the fluoroscopic image.

FIG. 2F is a diagram illustrating a relation between the rotational angle of the intra-medullary rod and the distance to the intersection of a pair of steel wires.

Suppose that a length of the cylindrical body 13 in the axial direction is 90 mm and that the intra-medullary rod is located at its initial position. The initial location of the intersection of a pair of steel wires, in other words, the reference position (origin), is at the center X0 of the cylindrical body 13. By digitizing both ends of the intra-medullary rod on the C-arm fluoroscopic image in this state, the coordinate system, exclusive of the rotation, of the intra-medullary rod is determined.

If the intra-medullary rod in this state rotates +15 degrees, the location of the intersection X1 of a pair of wires shifts to the right side by 15 mm from the origin, and if the intra-medullary rod in this state rotates −15 degrees, the location of the intersection X1 of a pair of wires shifts to the left side by 15 mm from the origin. Likewise, if the intra-medullary rod rotates +30 degrees, the location of the intersection X2 shifts to the right side by 30 mm, and if the intra-medullary rod rotates −30 degrees, the location of the intersection X2 shifts to the left side by 30 mm. If the intra-medullary rod rotates +45 degrees, the location of the intersection shifts to the right side by 45 mm, and if the intra-medullary rod rotates −45 degrees, the location of the intersection shifts to the left side by 45 mm. As such, by calculating a distance in the axial direction from the origin of the intra-medullary rod to the location of an intersection Xn of a pair of wires, the rotational angle of the intra-medullary rod can be obtained directly.

Thus, by digitizing the intersection of a pair of steel wires on a patient's C-arm fluoroscopic image, one can measure the rotational angle of the intra-medullary rod proportional to the axial-direction distance with respect to the origin.

Moreover, the number of wires 15 set up in the cylindrical body is not limited to 4, but it can be 3 at 120-degree intervals, 6 at 60-degree intervals, or 8 at 45-degree intervals. Besides, the length of the cylindrical body 13 may also be suitably designated according to its application or the number of wires 15, etc. Desirably, the outer diameter of the cylindrical body varies within a range of 6-10 mm, and the diameter of the wire varies within a range of 0.8-1.2 mm.

As in this embodiment, if the cylindrical body 13 is 90 mm long and four wires are set at a 90-degree intervals, there is an advantage that the relation between the angle and the distance to the intersection is explicitly simplified to a one-to-one correspondence. Depending on the length of a cylindrical body being used or the measurement unit (mm or inch, for example) thereof, the relation between the angle and the length can be set to be visibly simple.

Another feature of the present invention lies in its usage of the C-arm fluoroscopic apparatus 40. To provide an intraoperative assistance using the 3-dimensional limb alignment evaluation system, it is important to unify the environment for taking a clinical picture. Photographing with the C-arm fluoroscopic apparatus is carried out by moving a fluoroscopic apparatus to a position where the distance from a televising unit to an object to be photographed is approximately the same from two (the front and the lateral) directions. In this manner, the environment for photographing a clinical picture can be unified.

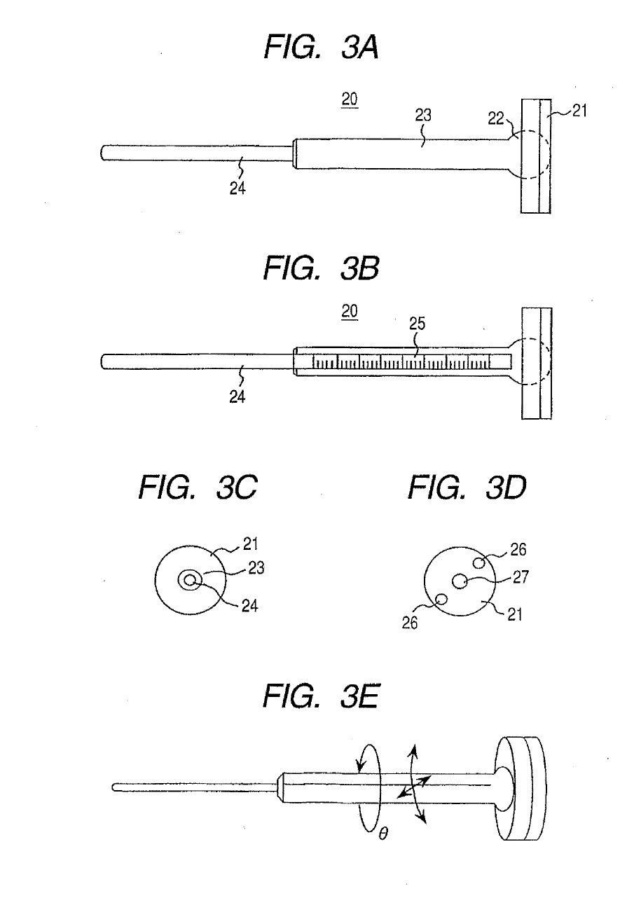

Next, a construction of the universal joint 20 indicating the bone cutting direction will be explained referring to FIG. 3. The universal joint 20 includes a base 21, a direction indicating jig 23 with a circular cross section in which a one surface having a ball joint 22 formed on one end is planar, a front end 24 with a circular cross section, a pair of holes 26, 26 formed in the base 21 into which pins on the far end of the intra-medullary rod 10 are inserted, and a screw 27 for fixing a positional relation between the ball joint 22 and the base 21. The direction indicating jig 23 has calibrations 25 for recognizing the position of the resected bone surface in the remote and near directions. Further, a bone cutting jig with a slit for guiding a bone saw is mounted on the direction indicating jig 23 of the universal joint 20.

The direction indicating jig 23 of the universal joint 20 is supported by the ball joint 22 in a manner that it can move/rotate around three axes about the base 21.

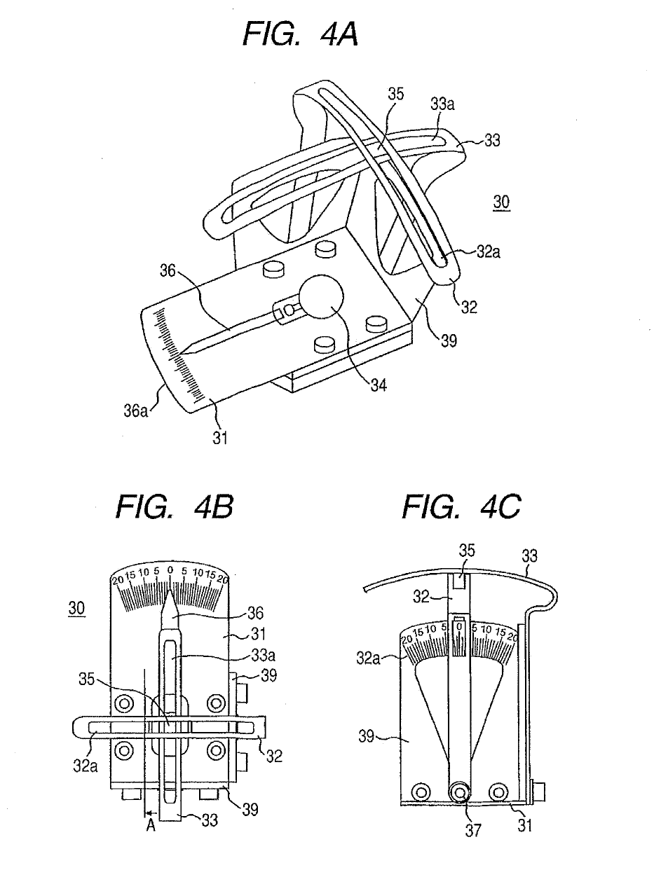

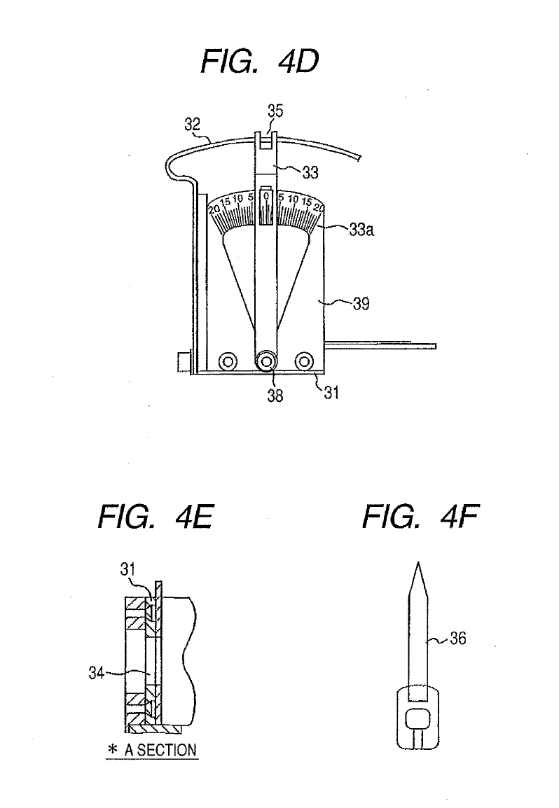

Next, FIG. 4 shows an example of the construction of the bone cutting direction indicator 30.

The bone cutting direction indicator 30 has a base 31, a sickle-shaped guide 32 attached to the base 31 through a shaft 37, which has a guide groove 32a formed on an upper surface and determines an inversion and evagination angle, and a sickle-shaped guide 32 attached to the base 31 through a shaft 38, which has a guide groove 33a formed on an upper surface and determines a bending and stretching angle. In addition, the direction indicating jig 23 of the universal joint 20 is inserted upwardly into a hole 34 of the base 31. The front end of the direction indicating jig 23 of the universal joint 20 is inserted into an intersection 35 of the guide 32 for determining the inversion and evagination angle and the guide 33 for determining the bending and stretching angle, and the bone cutting direction (angle) is determined by this direction indicating jig 23. Furthermore, side walls 39, 39 are screwed onto the base 31 in the inner side of the respective guides 32 and 33. The side walls 39, 39 have calibrations 32a and 33a, each showing an angle of the guide 32 and the guide 33.

An indicator probe 36 is installed at the base 31 to be rotatable around the hole 34, and the base 31 has calibrations 36a corresponding to the front end of the indicator probe 36. The calibrations 36a indicate internal/external rotation angles of the direction indicating jig 23.

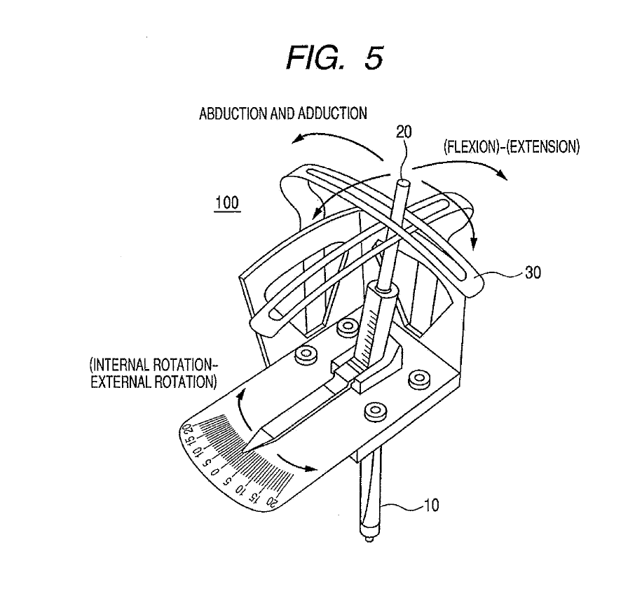

The following will now describe the overall construction and operation of the bone cutting positioning jig 100 with reference to FIG. 5.

As depicted in FIG. 5, the bone cutting positioning jig 100 includes the intra-medullary rod 10 and the bone cutting direction indicator 30 connected thereto through the universal joint 20.

The internal/external rotation angles of the intra-medullary rod 10 are indicated by the calibrations 36a of the direction indicating jig 23. It is possible to show the inversion and evagination angles by moving the guide 32 for determining the inversion and evagination angles. Moreover, it is possible to show the bending and stretching angles by moving the guide 33 for determining the bending and stretching angles.

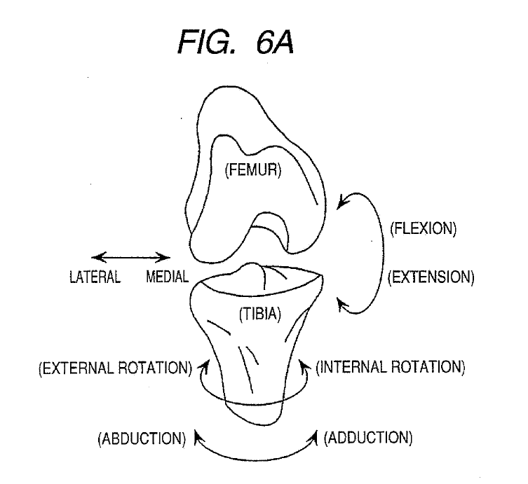

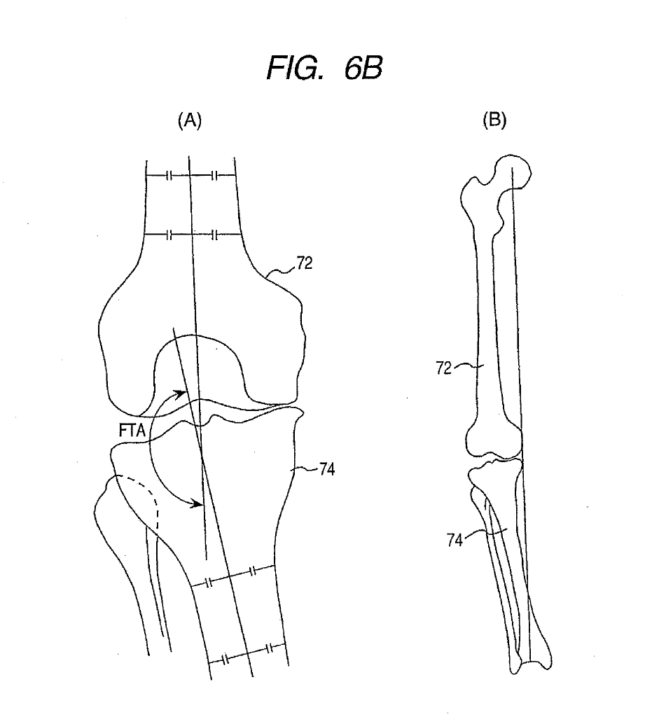

Next, a knee joint will be explained referring to FIG. 6. At first, a relation between a shape of the knee joint and a movement thereof is discussed in reference to FIG. 6A. The knee joint, by its structure, can be divided into two joints: a femoro-tibial joint formed of a femur 72 and a tibia 74 (refer to FIG. 6B), and a patella-femoral joint formed of a femur and a sesamoid bone. In addition, for the knee joint to support one's body weight as well as to ensure a stable movement, the femoro-tibial joint is not supported by an interactive joint between bones but by a joint of soft tissues inclusive of a strong, extendible joint capsule, ligament, tendon and the like.

The anatomical movement of the knee joint consists of three rotations, bending and stretching as the main one as indicated by arrows in FIG. 6A, internal and external rotations in the frontal plane, and abduction and adduction in the cross sectional faces, and three translational movements in inward/outward, front/rear, and remote-near directions.

The evaluation of limb alignment is important for an examination of load on the knee joint. As an example of its clinical indication, a femoro-tibial angle (FTA) and a functional axis are used. In general, a pedestrian standing lo leg period is assumed, a long film is used for one leg standing position, and X-ray images in the vicinity of the knee are used for measurement. A line connecting the center of the bone head of the femur and the center of the leg joint is designated as a limb functional axis (Mukulicz line), and a passing point of this knee joint becomes an index of the loaded state (FIG. 6B(b)).

Because of this, it is desired to do the limb alignment evaluation 3-dimensionally.

Meanwhile, the total knee joint is constituted by a tibial component and a tibial insert besides the femoral component, and by inserting the tibial insert between the femoral component made of a metallic material and the tibial component, abrasion of the total knee joint can be reduced.

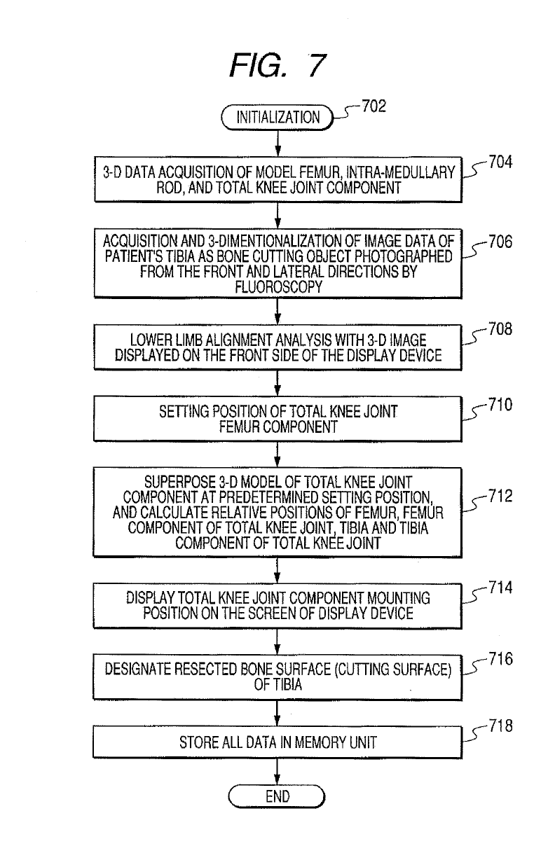

Referring to FIGS. 7-10, the following will now explain an operative assistance procedure using the total knee joint replacement assisting system of the present invention.

The operative assistance is divided into the planning assistance prior to the intervention and the intraoperative assistance, which is performed by utilizing the planning assistance function 202 prior to the intervention and the intraoperative assistance function 204 of the total knee joint replacement assisting terminal 200. For the intraoperative assistance, a bone cutting positioning jig is used.

First of all, the planning assistance function prior to the intervention is explained. FIG. 7 is a flow chart describing the planning assistance function prior to the intervention.

Initialization is performed at first, and an environment for making a plan prior to the intervention is created by using the total knee joint replacement assisting terminal 200 (S702).

Next, data on a femoral model, a rod on the drawing and 3-dimensional model of a total component (implant) are acquired from the database 270 of the total knee joint replacement assisting terminal 200 (S704).

Images are photographed in two directions, front and lateral, with the C-arm fluoroscopic apparatus. Using the C-arm radiographic apparatus and the CT scanner, an X-ray image and a CT image of the lesion of a patient's tibia are taken, and the radiographic image data and the CT image data are read by a computer, thereby 3-dimensionalizing the bone shape.

In addition, the photographed images have distortions. Thus, to correct the distortions of the images, a 3-dimensional polynomial approximation is used, and a calibration coefficient is calculated to express an accurate grid (S706).

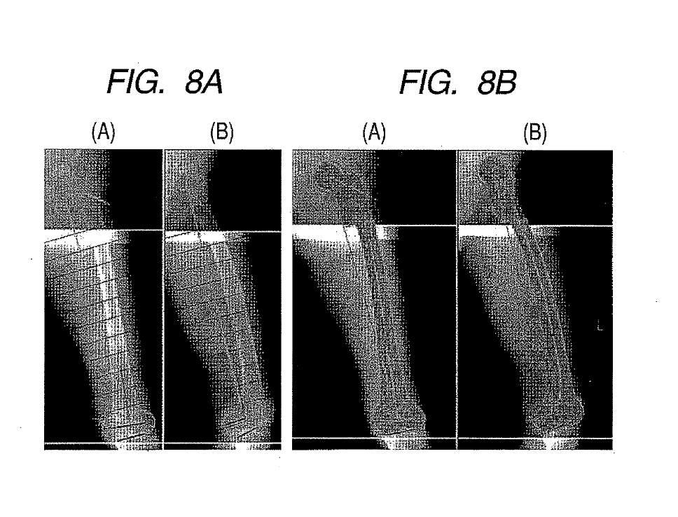

Later, a 3-dimensional limb alignment analysis is performed. That is, as shown in FIG. 8A, a femoral computer model is read on the screen, and a reference point for establishing a coordinate system of the bone head center, the medial and lateral posterior condyle center and the like is digitized. Further, a coordinate system for the tibial side is established by digitizing the medial, tubercle therebetween, anterior articular surface, etc. Then, as shown in FIG. 8B, the limb alignment analysis is performed using the 3-dimensional limb alignment evaluation system. By this work, an anteversional angle of an object to be analyzed or the limb alignment such as FTA is calculated (S708).

Afterwards, the setting position of the artificial knee joint femoral component is determined using a result of the 3-mensional limb alignment analysis (S710).

Next, prepared total knee joint component's computer models are read on the screen, and are superposed with each other at a predetermined setting position. Based on the radiographic image data and the shape data of an implant to be implanted, a 3-dimensional simulation is performed for setting the implant.

By this operation, relative positions of the femur and femoral component of the total knee joint, and relative positions of the tibia and tibial component of the total knee joint are calculated (S712).

A resected bone surface of the tibia is then set. That is, to determine an ideal mount position of an implant (resected bone surface of the tibia), a 3-dimensional simulation of the implant mount is performed. To this end, the radiographic image data, the CT image data and the implant shape data are displayed on the display unit (S714). First, the CT image seen from the anatomical axial direction (pelvis side) of the tibia is displayed on the display device 280, the kind of the implant to be used and the horizontal location of the resected bone surface are determined, and the determined position is outputted as the horizontal location data of the resected bone surface. Next, the determined horizontal location of the incisal surface is modified according to the radiographic image data and the CT image data. If the modification is needed, the implant makes a parallel movement back and forth or makes a rotational movement and the modified location is outputted as a 3-dimensional location direction data of the resected bone surface. This modified 3-dimensional location of the resected bone surface is also modified according to the radiographic image data seen laterally, and this modified location is outputted as a 3-dimensional location direction data of the resected bone surface. And, the 3-dimensional location direction data of the resected bone surface is stored in a memory device such as the database 270, thereby completing the process prior to the intervention (S718).

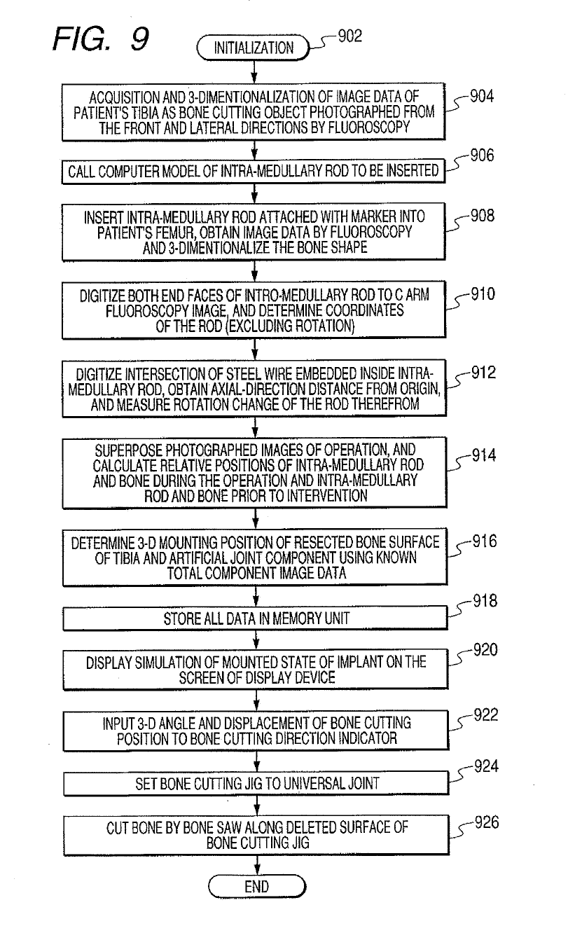

Referring now to FIG. 9, the intraoperative assistance function of the total knee replacement method is explained below.

Initialization is performed at first, and an environment for making a plan prior to the intervention is created by using the total knee joint replacement assisting terminal 200 (S902).

Using the C-arm radiographic apparatus and the CT scanner, an X-ray image and a CT image of the lesion of a patient's tibia are taken, and the radiographic image data and the CT image data are read by a computer, thereby storing it in the memory device and 3-dimensionalizing the bone shape at the same time (S904).

Then a computer model data on the intra-medullary rod 10 to be inserted is read from the database 270 (S906).

Next, soft tissues such as skin, muscle, joint capsule, and ligament are treated, and the resection of a patient's distal femoral joint surface is performed using a method called Intra-Medullary. For the resection of the head of femur, a hole is first drilled and the intra-medullary rod 10 is inserted therethrough into a femoral medullary cavity to determine an anatomical axis of the femur. That is, the intra-medullary rod 10 is inserted into the tibia of the lesion, and X-ray images of the tibia are photographed using the C-arm fluoroscopy apparatus 40 in two directions, front and lateral. The images thusly photographed are transmitted to a computer where they are subjected to distortion correction, and the shape of the bone is 3-dimensionalized (S908).

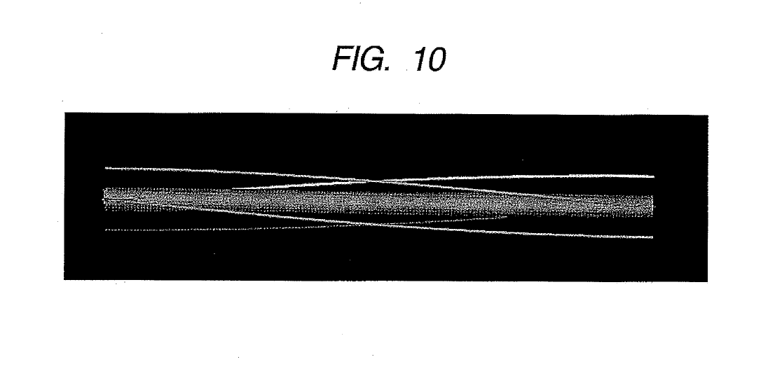

FIG. 10 shows an example of the C-arm fluoroscopic image of the intra-medullary rod.

A coordinate system (exclusive of rotation) of the intra-medullary rod is determined by digitizing both ends (long diameter and sectional end surface) of the intra-medullary rod on the C-arm fluoroscopic image (S910).

Later, an intersection of a pair of steel wires buried in the cylindrical body of the intra-medullary rod is digitized to obtain a distance from the origin in the axial direction, and a rotational angle of the rod is obtained therefrom (S912).

A resected bone surface about the direction of the rod is calculated by the location alignment with the planning before the intervention. Namely, a computer model of the intra-medullary rod to be inserted during an operation is called and is superposed on an image photographed during the operation, whereby the relative positions of the intra-medullary rod and bone during the operation and the relative positions of the intra-medullary rod and bone of the planning before the intervention are computed, respectively (S914).

A femoral computer model which maintained the relative position from the femur and the femoral component in the planning before the intervention is superposed on the image. By this operation, the relative position of the bone seen from the intra-medullary rod is determined. Using the shape data of a base total joint component, 3-dimensional mount positions of the resected bone surface of the tibia and total joint component are determined (S916).

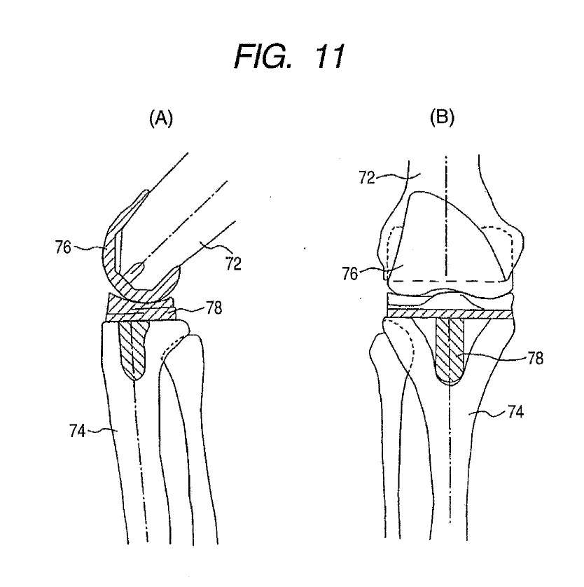

FIG. 11 illustrates a mount position of the total joint component (implant). Reference numeral 72 denotes a femur, reference numeral 76 denotes a femoral component, and reference numeral 78 denotes a tibial component.

Various data thusly obtained are kept in a memory device of the computer (S918).

Next, the mount position of the implant (resected bone surface of the tibia) is displayed on the screen of the display device (S920). That is to say, the 3-dimensional angle and displacement of the bone cutting position seen from the intra-medullary rod are displayed on the computer screen as digits.

Provided that the bone cutting jig 60 is mounted on the bone cutting positioning jig 100, an operator, based on the digits, adjusts an angle of the guide 32 for determining the inversion and evagination angles and an angle of the guide 33 for determining the bending and stretching angles of the bone cutting direction indicator 30, and sets an angle of the bone cutting jig 60 (S924). That is, using the rod 10 inserted into the medullary cavity as an anatomical axis, resection margins of the anterior articular surface of the patient's femur, the front of the head of the femur and the like are determined.

The operator, using the bone saw, resects the bone at the mount position of the implant, along the resection margin set by the bone cutting jig 60 (S926).

### INDUSTRIAL APPLICABILITY

According to the present invention, it becomes possible to accurately recognize the direction of a rod from a narrow field of vision for fluoroscopy, by using the special bone cutting positioning jig employing the intra-medullary rod.

That is, by using the special bone cutting positioning jig employing the intra-medullary rod, it becomes possible to calculate the direction and amount of the back and forth, lateral and far-to-near movement of the bone cutting jig about the resected bone surface in the planning before the intervention. Only medical instruments available in normal medical facilities are used, leading to reduction in operative time, and simplification of surgical instruments to be cleaned.

In addition, the intra-medullary rod 10 of the present invention can generate excellent effects when incorporated with the C-arm fluoroscopic apparatus.

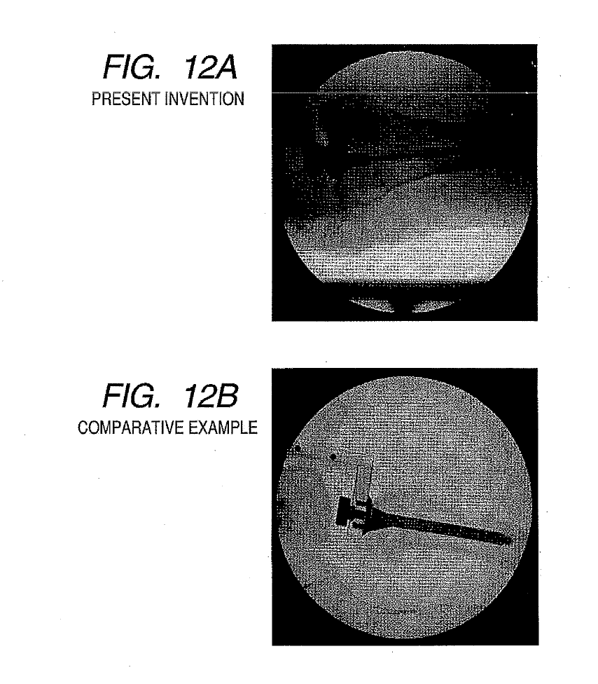

Referring to FIG. 12, one of the effects of the present invention, i.e., the capability of accurately recognizing the information on the intra-medullary rod from a narrow field of vision for fluoroscopy will now be explained.

In general, the C-arm fluoroscopic apparatus has a narrow field of vision for fluoroscopy, so that an error often occurs in the axial direction of the bone. Therefore, the setting position of the artificial knee joint femoral component before and after the intervention has a serious error in parameters exclusive of abduction and adduction, and the setting position is not stable. Meanwhile, these are contradictory problems to the simplification of the photographing environment creation and the improvement of accuracy in 3-dimensional re-establishment when the 2-directional photographing is performed clinically.

A comparative example shown in FIG. 12B is mounted with a member besides the intra-medullary rod, having a marker indicator function. According to this comparative example, although the C-arm fluoroscopic apparatus was used, since the field of vision for fluoroscopy is narrow, an error often occurs in the axial direction of the bone. The setting of the artificial knee joint femoral component before and after the intervention has a serious error in parameters exclusive of abduction and adduction, and is not stable.

On the other hand, in case of the method of the present invention shown in FIG. 12A, since the intra-medullary rod 10 has the marker indicator function, the field of vision for fluoroscopy is broad and errors are less common in the axial direction of the bone.

Therefore, the intra-medullary rod 10 of the present invention is desirable to be photographed by the C-arm fluoroscopic apparatus. Since the C-arm fluoroscopic apparatus is easily controlled to 0-90 degrees, the X-ray radiation point and the televised unit are in one-to-one correspondence and the distance between the X-ray radiation point and the televised unit is fixed all the time. These features are sufficient to overcome all problems in the intraoperative photographing which uses a 0-90 degree cassette table. Moreover, by utilizing the C-arm fluoroscopic apparatus for intraoperative photographing, the clinical photographing task is less difficult. When the C-arm fluoroscopic apparatus is used, it takes about 5 minutes from the intraoperative photographing to the indication of bone cutting position, which leads to the reduction in operative time and the precise bone cutting.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. An intra-medullary rod, comprising:
a cylindrical body made of an X-ray transmitting material;
a plurality of wires, which are made of a material that does not transmit X-ray and are deposited at regular intervals along a surface of the cylindrical body in the circumferential direction, being extended in an axial direction in a spiral shape,
wherein each of the wires is made in a way that a starting end and a terminating end of the cylindrical body are connected by the shortest distance along the outer surface thereof.

2. An intra-medullary rod comprising a cylindrical body made of a non-metallic material and a plurality of wires in a spiral shape being formed on an outer surface of the cylindrical body at regular intervals, wherein provided that a first circle and a second circle at both ends of the cylindrical body each having a same diameter corresponding to the surface portion are formed, a starting end of each of the wires is located at regular intervals on the first circle, a terminating end of each of the wires is at a position on the second circle rotated in predetermined-degree increments from the starting end, and each of the wires connects the starting end and the terminating end of the cylindrical body by a straight line when the cylindrical body is developed on a plane.

3. The intra-medullary rod according to claim 1 or claim 2, wherein the cylindrical body is made of an acrylic resin and each of the wires is made of stainless steel.

4. An intra-medullary rod according to claim 1 or claim 2, wherein the cylindrical body is a hollow cylindrical body having a metallic core material at the center, and the core material is connected to an end of the metallic material deposited on both ends of the outside of the cylindrical body.

5. An intra-medullary rod, comprising: a cylindrical body of which central portion except for both ends is made of an X-ray transmitting material, and a plurality of spiral-shaped wires made of a material that does not transmit X-rays and being installed on a surface of the cylindrical body at regular intervals, each of the wires connecting a starting end and a terminating end of the cylindrical body by the shortest distance, and
wherein, in a fluoroscopic image of the cylindrical body, a distance from an origin to an intersection of the wires is measured by an amount of rotational angle of the intra-medullary rod.

6. The intra-medullary rod according to one of claims 1-5, wherein a length of the cylindrical body is 90 mm and 4 stainless steel wires are installed at intervals of 90 degrees along an outer surface portion of the cylindrical body.

7. A bone cutting positioning jig for indicating a bone cutting direction, comprising: a bone cutting direction indicator having a base, a universal joint being movably and rotatably supported around three axes to the base of the bone cutting direction indicator through a ball joint and having a direction indicating jig, and an intra-medullary rod fixed to one end of the universal joint,
wherein the intra-medullary rod comprises a cylindrical body made of a non-metallic material and a plurality of wires in a spiral shape being formed on an outer surface of the cylindrical body at regular intervals, an intersection of each of the wires having a marker indicator function providing rotational position information;
wherein the bone cutting direction indicator comprises a guide having a guide groove on an upper surface and being attached to the base through a shaft for determining an inversion and evagination angle, and a guide having a guide groove on an upper surface and being attached to the base through a shaft for determining a bending and stretching angle;
wherein a front end of a direction indicating jig of the universal joint is inserted into an intersection of the groove of the guide for determining an inversion and evagination angle and the groove of the guide for determining a bending and stretching angle; and
wherein the bone cutting direction is determined by the direction indicating jig.

8. A bone cutting positioning jig for indicating a bone cutting direction, comprising: a bone cutting direction indicator having a base, a universal joint being movably rotatably supported around 3 axes to the base of the bone cutting direction indicator through a ball joint and having a direction indicating jig, and an intra-medullary rod fixed to one end of the universal joint,
wherein the intra-medullary rod comprises a cylindrical body made of a non-metallic material and a plurality of wires in a spiral shape being formed on an outer surface of the cylindrical body at regular intervals, in which, provided that a first circle and a second circle at both ends of the cylindrical body each having a same diameter corresponding to the surface portion are formed, a starting end of each of the wires is located at regular intervals on the first circle, a terminating end of each of the wires is at a position on the second circle rotated in predetermined-degree increments from the starting end, each of the wires connects the starting end and the terminating end of the cylindrical body by a straight line when the cylindrical body is developed on a plane, and an intersection of each of the wires has a marker indicator function providing rotational position information;
wherein the bone cutting direction indicator comprises a guide having a guide groove on an upper surface and being attached to the base through a shaft for determining an inversion and evagination angle, and a guide having a guide groove on an upper surface and being attached to the base through a shaft for determining a bending and stretching angle;
wherein a front end of a direction indicating jig of the universal joint is inserted into an intersection of the groove of the guide for determining an inversion and evagination angle and the groove of the guide for determining a bending and stretching angle; and
wherein the bone cutting direction is determined by moving the inversion and evagination angle-determining guide of the direction indicating jig to indicate the inversion and evagination angles and by moving the bending and stretching angle-determining guide to indicate the bending and stretching angles.

9. A total knee joint replacing operation assisting terminal, constituted by a computer configured to assist a total knee joint replacing operation by performing a planning assistance function and an intraoperative assistance function;
wherein the planning assistance function is used before an intervention on a patient, and the intraoperative assistance function is used during the intervention on a patient, whereby the terminal is configured to assist a the total knee joint replacing operation performed with aid of a bone cutting positioning jig indicating a bone cutting direction by the intraoperative assistance function;
wherein the bone cutting positioning jig comprises a bone cutting direction indicator having a base, a universal joint being movably and rotatably supported around three axes to the base of the bone cutting direction indicator through a ball joint and having a direction indicating jig, and an intra-medullary rod fixed to one end of the universal joint;
wherein the intra-medullary rod comprises, a cylindrical body made of an X-ray transmitting material fixed at a central portion between a pair of both ends of the intra-medullary rod, a plurality of wires, which are made of a material that does not transmit X-ray and are deposited at regular intervals along a surface of the cylindrical body in the circumferential direction, being extended in an axial direction in a spiral shape;
wherein an intersection of each of the wires having a marker indicator function providing rotational position information;
wherein each of the wires is made in a way that a starting end and a terminating end of the cylindrical body are connected by the shortest distance along the outer surface thereof;
wherein the planning assistance function includes:
acquiring a load shaft of a patient's foot from 3-dimensional radiographic image data, and storing computer model data, on the intra-medullary rod to be inserted into a medullary cavity, in a database;
wherein the intraoperative assistance function includes:
acquiring, from the database, the computer model data on the intra-medullary rod to be inserted;
reading the computer model data, acquired from the database, on the intra-medullary rod to be inserted;
acquiring, by a C-arm fluoroscopic apparatus, radiographic image data of the intra-medullary rod inserted into a tibia of the patient;
acquiring, on the radiographic image data obtained by the fluoroscopic apparatus, rotational position information of the intra-medullary rod in a medullary cavity of the patient based on the position of the intersection of the pair of wires of the intra-medullary rod; and
determining a resected bone surface using the intra-medullary rod as a reference anatomical axis; and
wherein an anterior articular surface of the femur is determined perpendicularly to the load shaft of the patient from an angle with the femoral load shaft determined using the planning assistance function before the intervention, and a bone surface to be resected is determined.

10. The total knee joint replacing operation assisting terminal of claim 9, wherein the planning assistance function further includes performing a 3-dimensional simulation for mounting an artificial knee implant based on the 3-dimensional radiographic image data.

11. The total knee joint replacing operation assisting terminal of claim 10, wherein the planning assistance function further includes determining a kind of artificial knee implant to be used.

12. The total knee joint replacing operation assisting terminal of claim 11, wherein the planning assistance function further includes determining a setting position of the artificial knee implant.

13. The total knee joint replacing operation assisting terminal of claim 12, wherein the planning assistance function further includes superposing the 3-dimensional radiographic image data with the setting position of the artificial knee implant.

14. The total knee joint replacing operation assisting terminal of claim 9, wherein the intraoperative assistance function further includes superposing the computer model data on the intra-medullary rod to be inserted on the radiographic image data acquired during the intraoperative assistance function.

15. The total knee joint replacing operation assisting terminal of claim 9, wherein the intraoperative assistance function further includes resecting a bone surface using the bone cutting positioning jig and the determined resected bone surface.

16. The total knee joint replacing operation assisting terminal of claim 9, wherein the radiographic image data is a fluoroscopic image.
